(19) United States
(12) Patent Application Publication
(10) Pub. No.: US 2026/0130812 A1
(43) Pub. Date: May 14, 2026

(54) Orthotic Device Cuff Assemblies

(71) Applicant: Myomo, Inc., Boston, MA (US)

(72) Inventors: Divya Krishnagiri, Cambridge, MA (US); Andrew Harlan, Somerville, MA (US); Jake A. Schwartz, Boston, MA (US); Dan Echeverria, Cambridge, MA (US)

(21) Appl. No.: 18/941,309

(22) Filed: Nov. 8, 2024

(51) Int. Cl. A61H 1/02

(52) U.S. Cl. CPC A61H 1/0277; A61H 2001/0207; A61H 2201/1638; A61H 2201/1676; A61H 2205/06; A61H 2230/085

(57) ABSTRACT

An example of an asymmetric cuff assembly for a powered orthotic device for use by a wearer includes: a first cuff shell having a first concave interior surface shaped to overlie a first muscle of a limb of the wearer; a second cuff shell having a second concave interior surface shaped to overlie a second muscle of the limb of the wearer and having a second cross-sectional area that is greater than a first cross-sectional area of the first cuff shell; and a pivot mechanism pivotally coupling the first cuff shell to the second cuff shell along an axis that is generally parallel to the limb of the wearer with the asymmetrical cuff assembly worn by the wearer.

### BACKGROUND

Stroke, brain injury, and other neuromuscular trauma survivors are often left with hemiparesis, or severe weakness, in certain parts of the body. This may result in impaired or lost function in one or more limbs. It has been shown that neuromuscular trauma survivors can rehabilitate significantly from many of the impairments following such neuromuscular traumas by, for example, following a rehabilitative exercise regime that includes the execution of familiar and functional tasks. Many neuromuscular trauma survivors may use powered orthotic devices to assist and/or enhance their abilities to perform these tasks. If, however, a powered orthotic device is difficult to don and/or uncomfortable to wear and use, a neuromuscular trauma survivor will likely use the powered orthotic device less, and thus realize less rehabilitative benefit than may otherwise be achieved.

### SUMMARY

An example of a cuff system for use in an orthotic device having a muscle sensor system includes: a cuff shell that is at least semi-rigid and has a concave cross section, in a plane transverse to a length of the cuff shell, to accommodate attachment of the cuff shell to a limb; a fabric liner disposed in the cuff shell; and a sensor array including a set of sensor contacts, in coordination with the fabric liner, configured to attach to the fabric liner in alignment with a discontinuity of the cuff shell.

Implementations of such a cuff system may include at least one of the following features. The discontinuity is a recess in the cuff shell. The cuff system includes a bias member disposed between the fabric liner and the cuff shell at least partially in the recess. The bias member includes a foam pad.

Also or alternatively, implementations of such a cuff system may include at least one of the following features. The discontinuity is an opening through the cuff shell. The fabric liner includes a plurality of slits with each slit being transverse to an axial direction of the cuff shell. Each slit of the plurality of slits is disposed proximate to a respective side of the discontinuity of the cuff shell.

Also or alternatively, implementations of such a cuff system may include at least one of the following features. The cuff shell is a first cuff shell, the sensor array is a first sensor array, the discontinuity of the cuff shell is a first discontinuity of the first cuff shell, and the cuff system includes: a second cuff shell pivotally coupled to the first cuff shell; and a second sensor array attached to the fabric liner in alignment with a second discontinuity of the second cuff shell. The cuff shell is a first cuff shell, the fabric liner is a first fabric liner, the sensor array is a first sensor array, the concave cross section of the first cuff shell is configured to accommodate attachment of the first cuff shell to a first portion of the limb, and the cuff system includes: a second cuff shell that is at least semi-rigid and has a concave cross section, in a plane transverse to a length of the second cuff shell, to accommodate attachment of the second cuff shell to a second portion of the limb; a second fabric liner disposed in the second cuff shell; and a second sensor array attached to the fabric liner in alignment with a discontinuity of the second cuff shell. The concave cross section of the first cuff shell is configured to accommodate attachment of the first cuff shell to a forearm of a user and the concave cross section of the second cuff shell is configured to accommodate attachment of the second cuff shell to an upper arm of the user.

Also or alternatively, implementations of such a cuff system may include at least one of the following features. An area of the sensor array is smaller than an area of the discontinuity. The sensor array is configured to releasably attach to the fabric liner. The cuff shell includes a recess along a portion of a length of the cuff shell to receive an electrical cable connected to the sensor array. The fabric liner includes an elasticized material. The fabric liner includes a rubber foam.

An example of an asymmetric cuff assembly for a powered orthotic device for use by a wearer includes: a first cuff shell having a first concave interior surface shaped to overlie a first muscle of a limb of the wearer; a second cuff shell having a second concave interior surface shaped to overlie a second muscle of the limb of the wearer and having a second cross-sectional area that is greater than a first cross-sectional area of the first cuff shell; and a pivot mechanism pivotally coupling the first cuff shell to the second cuff shell along an axis that is generally parallel to the limb of the wearer with the asymmetrical cuff assembly worn by the wearer.

Implementations of such an asymmetric cuff assembly may include at least one of the following features. The pivot mechanism is configured to support an open state of the cuff shells for donning and removing of the asymmetric cuff assembly, and a plurality of limb-containment states of the cuff shells, and wherein the pivot mechanism is configured to inhibit relative angular motion of the first cuff shell and second cuff shell about the axis with the cuff shells in the open state. The pivot mechanism includes a tapered detent to inhibit pivoting of the first cuff shell relative to the second cuff shell with the asymmetric cuff assembly in the open state.

Also or alternatively, implementations of such an asymmetric cuff assembly may include at least one of the following features. The pivot mechanism includes at least one first cuff pivot member that is integral with the first cuff shell and at least one second cuff pivot member that is integral with the second cuff shell. At least one of the first cuff shell and the second cuff shell is semi-rigid. The asymmetric cuff assembly includes a retention mechanism to hold the asymmetric cuff assembly in a selected one of the plurality of limb-containment states, the retention mechanism including: a strap coupled to the second cuff shell; and a ring pivotally coupled to the first cuff shell to pivot between a donning state and an in-use state, the ring extending away from the first cuff shell more in the donning state than in the in-use state, at least one of the first cuff shell and the ring configured to inhibit pivoting of the ring with the ring in the donning state; wherein the strap includes a first portion configured to pass through the ring and to releasably attach to a second portion of the strap. The ring extends away from the first cuff shell in the in-use state to allow the strap to slide between the ring and the first cuff shell.

Also or alternatively, implementations of such an asymmetric cuff assembly may include at least one of the following features. The asymmetric cuff assembly includes a retention mechanism to hold the asymmetric cuff assembly in a selected one of the plurality of limb-containment states, the retention mechanism including: a ring coupled to the first cuff shell; and a fabric strap coupled to the second cuff shell and having an end portion that is configured to pass through the ring and that includes a strap stiffener that inhibits the fabric strap from drooping downward due to gravity. The asymmetric cuff assembly includes a retention mechanism to hold the asymmetric cuff assembly in a selected one of the plurality of limb-containment states, the retention mechanism including: a ring coupled to the first cuff shell; and a fabric strap including a base portion coupled to the second cuff shell and an end portion that is configured to pass through the ring and that includes an asymmetrical set of attachment jaws configured to attach to respective sides of the base portion. The asymmetric cuff assembly includes a retention mechanism to hold the asymmetric cuff assembly in a selected one of the plurality of limb-containment states, the retention mechanism including: a ring coupled to the first cuff shell; and a fabric strap coupled to the second cuff shell and including an end portion that is configured to pass through the ring and that includes an outer webbing member and an inner webbing with the inner webbing member attached to the outer webbing member at least two connection points, wherein a length of the inner webbing member between the two connection points is shorter than a length of the outer webbing member between the two connection points. The asymmetric cuff assembly is an asymmetric humeral cuff assembly wherein the first cuff shell is a biceps cuff shell configured to overlie a biceps of the wearer, and wherein the second cuff shell is a triceps cuff shell configured to overlie a triceps of the wearer.

An example of an arm cuff assembly for a powered orthotic device for use by a wearer includes a first cuff shell pivotally coupled to a second cuff shell, and a retention mechanism including: a fabric strap coupled to the first cuff shell; and a ring pivotally coupled to the second cuff shell; wherein at least one of: (1) the ring can pivot between a donning state and an in-use state, the ring extending away from the second cuff shell more in the donning state than in the in-use state, at least one of the second cuff shell and the ring configured to inhibit pivoting of the ring with the ring in the donning state, and the strap includes a first portion configured to pass through the ring and to releasably attach to a second portion of the strap; (2) the fabric strap has an end portion that is configured to pass through the ring, and the fabric strap has a base portion that includes a strap stiffener to cause the fabric strap to extend away from the first cuff shell; (3) the fabric strap includes a base portion coupled to the first cuff shell and an end portion that is configured to pass through the ring and that includes an asymmetrical set of attachment jaws configured to attach to respective sides of the base portion; and (4) the fabric strap includes an end portion that is configured to pass through the ring and that includes an outer webbing member and an inner webbing member with the inner webbing member attached to the outer webbing member at at least two connection points, wherein a length of the inner webbing member between the two connection points is shorter than a length of the outer webbing member between the two connection points.

Implementations of such an arm cuff assembly may include at least one of the following features. The ring extends away from the first cuff shell in the in-use state to allow the strap to slide between the ring and the first cuff shell.

### BRIEF DESCRIPTION OF THE DRAWINGS

FIG. 1 is a perspective view of an example of an orthotic device, according to an embodiment of the invention, with a humeral cuff assembly in an open state.

FIG. 2 is a perspective view of the orthotic device shown in FIG. 1, being worn by a wearer.

FIG. 3 is an end view of an example of an asymmetric humeral cuff of an example of an asymmetric cuff assembly shown in FIG. 1.

FIG. 4A is a plan view of the asymmetric humeral cuff shown in FIG. 3 in a non-open state.

FIG. 4B is a plan view of the asymmetric humeral cuff shown in FIG. 3 in an open state.

FIG. 5A is an end view of the asymmetric humeral cuff assembly shown in FIG. 1 in the open state.

FIG. 5B is an end view of the asymmetric humeral cuff assembly shown in FIG. 1 in an intermediate donning state.

FIG. 5C is an end view of the asymmetric humeral cuff assembly shown in FIG. 1 in a donned state.

FIG. 6 is a side plan view of an end portion of an example of a securing strap of the asymmetric humeral cuff assembly shown in FIG. 1.

FIG. 7, is a top plan view of the end portion of the securing strap of the asymmetric humeral cuff assembly shown in FIG. 1.

FIG. 8 is a block flow diagram of an example of a method of donning a powered orthotic device according to an embodiment of the invention.

FIG. 9 is a perspective view of a humeral cuff assembly shown in FIG. 1.

FIG. 10A is an exploded perspective view of components of the humeral cuff assembly shown in FIG. 9.

FIG. 10B is a perspective view of a triceps cuff shell shown in FIG. 10A.

FIG. 11A is a cross-section of the humeral cuff assembly shown in FIG. 9, worn by an upper arm of a user with the upper arm unflexed.

FIG. 11B is a cross-section of the humeral cuff assembly shown in FIG. 9, worn by an upper arm of a user with the upper arm flexed.

FIG. 12 is a cross-section of another example of a humeral cuff assembly, according to an embodiment of the invention, worn by an upper arm of a user with the upper arm flexed.

FIG. 13 is an end view, taken along line 13-13 in FIG. 1, of an asymmetric medial/lateral cuff, shown in FIG. 1, resting on a surface and disposed in an open position.

### DETAILED DESCRIPTION

Definitions. As used in this description and the accompanying claims, the following terms shall have the meanings indicated, unless the context otherwise requires.

A “set” has at least one element.

An “array” has at least one element.

“Semi-rigid” means capable of resiliently deflecting in response to force.

Techniques are discussed herein for facilitating donning and removal of an orthotic device, and retaining an arm in the orthotic device once donned. For example, a asymmetric humeral cuff assembly, of an orthotic device, includes an asymmetric humeral cuff for receiving an upper arm of a wearer of the orthotic device (which may be called a patient). The cuff may be opened, and when in an open state may resist closing. A strap for securing the cuff around the wearer's arm may be connected to a triceps cuff shell of the cuff assembly and include a strap that is semi-stiff such that the strap extends away from the triceps cuff shell, resisting gravity, to facilitate location and engagement (e.g., grasping, holding, hooking) by the wearer. The strap includes a stiffener to cause the strap to stand out from the cuff shell, inhibiting a portion of the strap from drooping downward due to gravity, such that the strap can be easily located and grasped. The strap may include a loop to facilitate engagement by the wearer. A ring may be coupled to a biceps cuff shell of the cuff assembly and may be held in a donning position that facilitates insertion of the strap through the ring. The strap may be inserted through the ring, looped back onto itself, and attached to itself (e.g., with a hook-and-loop fastener). Other techniques, however, may be used.

Techniques are also discussed herein for adapting to movement of a wearer of a powered orthotic device. For example, a cuff assembly for retaining a wearer's arm may include cuff shells pivotally connected to each other, a liner inside the cuff shells, and an array of one or more sensors attached to the liner. The liner may help retain the sensor array(s) against the wearer. One or more of the cuff shells may include a discontinuity that allows one or more sensor arrays to move outwardly (e.g., through an opening in an inner surface of the shell(s)) to accommodate movement (e.g., flexing of a muscle) of the wearer while maintaining contact between the sensor array(s) and skin of the wearer. Other techniques, however, may be used.

Items and/or techniques described herein may provide one or more of the following capabilities, as well as other capabilities not mentioned. One-hand donning and removal of an orthotic device by a wearer of the device, without assistance from another person, may be facilitated. An orthotic device may be more comfortable to be worn than previous orthotic devices. One or more sensors (e.g., one or more EMG (electromyographic) sensors) may be held comfortably in contact with the skin of a wearer of a powered orthotic device during movement (such as muscle flexing) of the wearer. A sensor pocket may be provided in a cuff shell of a cuff assembly of an orthotic device, with the sensor pocket facilitating flexing of the cuff shell while maintaining torsional stability of the cuff shell. One or more sensors may be gently held against the skin of the wearer while a cuff assembly is securely mounted to the wearer for stability. Other capabilities may be provided and not every implementation according to the disclosure must provide any, let alone all, of the capabilities discussed.

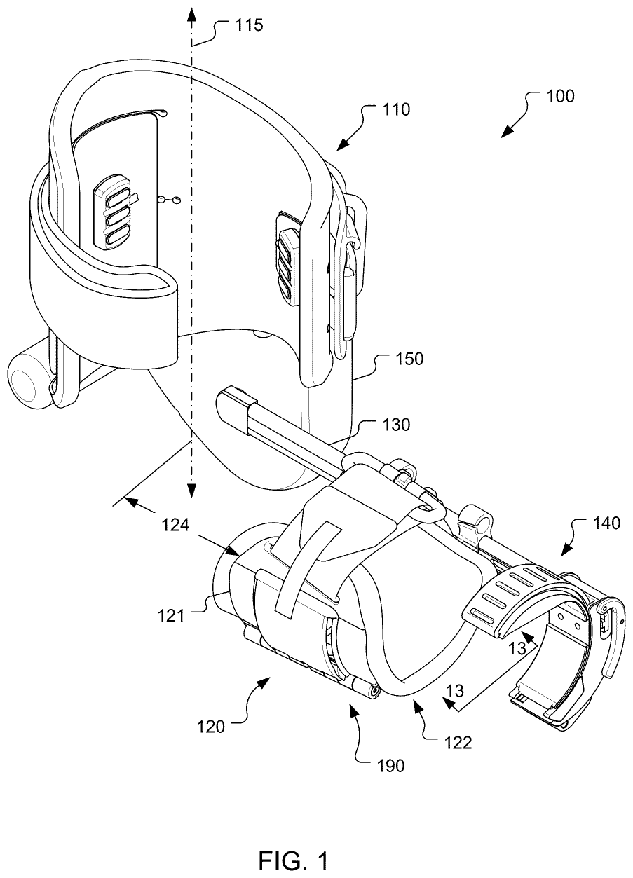

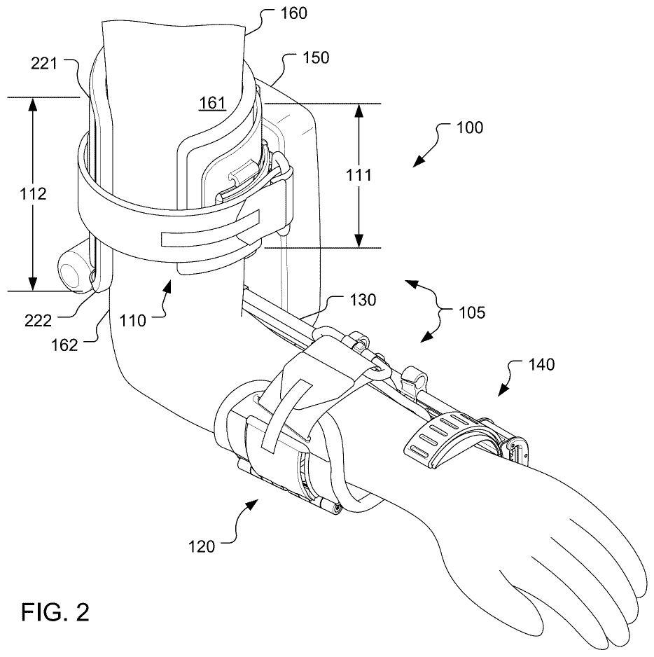

Referring to FIG. 1 and FIG. 2, a powered orthotic device 100 includes a cuff system 105 (including a humeral cuff assembly 110 and a medial/lateral cuff assembly 120), a support arm 130, a hand assembly 140, and an arm motor assembly 150. The humeral cuff assembly 110 is configured (e.g., sized, shaped, and equipped) to receive an upper arm 160 of a wearer of the device 100, to be wrapped around the upper arm 160, and to be secured around the upper arm 160 as shown in FIG. 2. The medial/lateral cuff assembly 120 is configured to receive a lower arm 170 of the wearer of the device 100, to be wrapped around the lower arm 170, and to be secured around the lower arm 170 as shown in FIG. 2. The support arm 130 connects the hand assembly 140 to the arm motor assembly 150. The hand assembly 140 is configured to move a hand of the wearer in response to electrical signals detected in the lower arm 170 of the wearer by sensors in the medial/lateral cuff assembly 120. The arm motor assembly 150 is configured to move the support arm 130 relative to the humeral cuff assembly 110, and thus the lower arm 170 relative to the upper arm 160, in response to electrical signals detected in the upper arm 160 of the wearer by sensors in the humeral cuff assembly 110. Other configurations of orthotic devices may be used. For example, a cuff system may include the humeral cuff assembly 110 but not the medial/lateral cuff assembly 120, or may include the medial/lateral cuff assembly 120 but not the humeral cuff assembly 110. Still other configurations may be used.

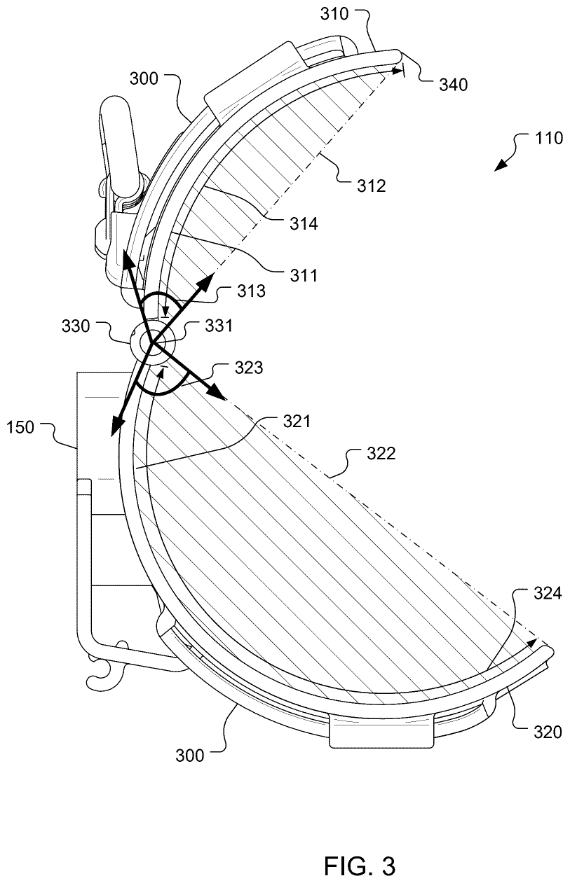

Referring also to FIG. 3, the humeral cuff assembly 110 includes a humeral cuff 300 that is shaped to receive and partially surround the upper arm 160. The humeral cuff 300 includes a biceps cuff shell 310 and a triceps cuff shell 320 pivotally coupled to each other by a pivot mechanism 330 about a pivot axis 331. The terms “triceps” and “biceps” include the singular and the plural, with the biceps cuff shell 310 configured to accommodate the biceps of a single arm and the triceps cuff shell 320 configured to accommodate the triceps of a single arm. The biceps cuff shell 310 has a concave interior surface 311 shaped to overlie a biceps 161 (see FIG. 2) of the upper arm 160 of the wearer. The concave interior surface 311 may be arcuate or another concave shape, and the concave shape may vary over a length 111 (see FIG. 1) of the biceps cuff shell 310. The concave interior surface 311 is concave transverse to the length 111 of the biceps cuff shell 310. The triceps cuff shell 320 has a concave interior surface 321 shaped to overlie a triceps 162 (see FIG. 2) of the upper arm 160 of the wearer. The concave interior surface 321 may be arcuate or another concave shape, and the concave shape may vary over a length 112 (see FIG. 1) of the triceps cuff shell 320. The concave interior surface 321 is concave transverse to the length 112 of the triceps cuff shell 320.

The humeral cuff 300 is asymmetrical, with the biceps cuff shell 310 and the triceps cuff shell 320 having different sizes. For example, a biceps cross-sectional area 312 of the biceps cuff shell 310 is smaller than a triceps cross-sectional area 322 of the triceps cuff shell 320, i.e., the triceps cross-sectional area 322 of the triceps cuff shell 320 is greater than the biceps cross-sectional area 312 of the biceps cuff shell 310. As another example, a biceps cuff shell sub-tended angle 313 (measured from a pivot axis 331) is smaller than a triceps cuff shell sub-tended angle 323 (measured from the pivot axis 331). As another example, a greatest width 314 of the biceps cuff shell 310 as measured along the interior surface 311 may be shorter than a greatest width 324 of the triceps cuff shell 320 as measured along the interior surface 321.

The humeral cuff 300 is sized to comfortably received and partially surround the upper arm 160 with a liner (discussed below) disposed in the humeral cuff 300. The humeral cuff 300 may be made in different sizes to accommodate different sizes of wearers. For example, Table 1 shows example sizes of different humeral cuffs.

TABLE 1

| Biceps cuff shell | Triceps cuff shell |
|---|---|
| circumferential width | circumferential width |
| 60 mm | 124 mm |
| 75 mm | 137 mm |
| 91 mm | 146 mm |

These sizes are examples and not limiting of the disclosure (including the claims), and other sizes of humeral cuffs may be used.

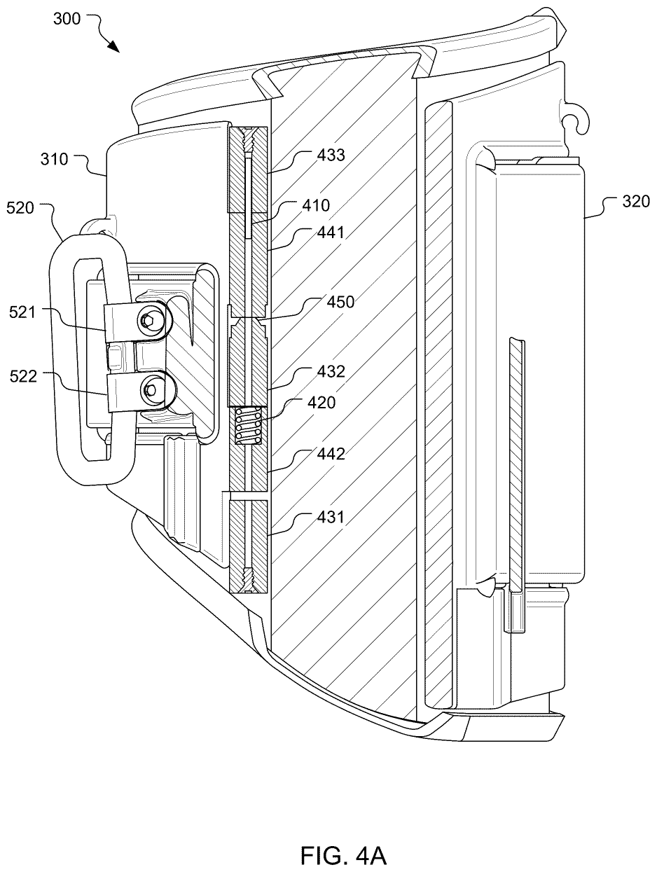

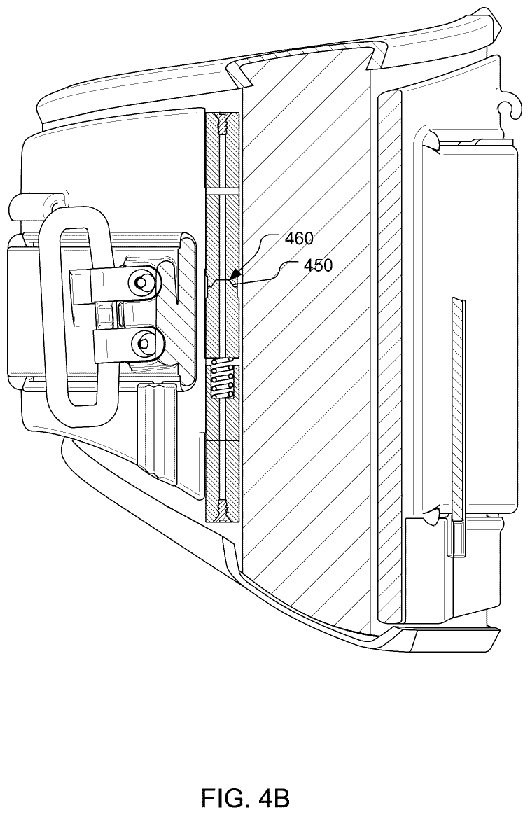

Referring also to FIG. 4A and FIG. 4B, the humeral cuff 300 includes the pivot mechanism 330 that pivotally couples the biceps cuff shell 310 to the triceps cuff shell 320 about the pivot axis 331. FIG. 4A shows the humeral cuff 300 in a non-open state (e.g., a secure state in which the humeral cuff 300 may be secured to a user or an intermediate state between an open state and a secure state). FIG. 4B shows the humeral cuff 300 in an open state in which the pivot mechanism 330 helps retain the humeral cuff 300 in the open state. The pivot axis 331 will be generally parallel (within 10° of parallel) to a humerus bone of the wearer of the device 100 with the asymmetrical humeral cuff 300 worn by the wearer (secured to the upper arm 160 of the wearer). The pivot axis 331 is generally parallel (within 10° of parallel) to the length 111 of the biceps cuff shell 310 and the length 112 of the triceps cuff shell 320.

The pivot mechanism 330 is configured to facilitate donning of the orthotic device 100 by the wearer without assistance. The pivot mechanism 330 is configured to enable the biceps cuff shell 310 to pivot relative to the triceps cuff shell 320. The pivot mechanism 330 is configured to support an open state of the cuff shells 310, 320, for donning and removing the asymmetrical humeral cuff 300, and multiple arm-containment states of the cuff shells 310, 320.

The pivot mechanism 330 is configured to inhibit relative angular motion of the biceps cuff shell 310 and the triceps cuff shell 320 about the pivot axis 331 with the cuff shells 310, 320 in the open state. In this example, the pivot mechanism 330 includes an axle 410, a spring 420, biceps cuff pivot members 431, 432, 433, and triceps cuff pivot members 441, 442. The biceps cuff pivot members 431, 432, 433 are integral with other portions of the biceps cuff shell 310, and the triceps cuff pivot members 441, 442 are integral with other portions of the triceps cuff shell 320. The biceps cuff pivot members 431, 432, 433 can pivot relative to the triceps cuff pivot members 441, 442 about the axle 410. The biceps cuff pivot members 431, 432, 433 can translate relative to the triceps cuff pivot members 441, 442 parallel to a length of the axle 410 (e.g., with the biceps cuff pivot members 431, 432, 433 and/or the triceps cuff pivot members 441, 442 sliding along a length of the axle 410). The spring 420 biases the biceps cuff pivot member 432 toward the triceps cuff pivot member 442. The biceps cuff pivot member 432 includes a tapered detent 450 (a cam) and the triceps cuff pivot member 441 includes a tapered recess 460 with a shape that approximately matches the shape of the tapered detent 450. The recess 460 is sized and shaped to receive the tapered detent 450 with the cuff shells 310, 320 in the open state. With the tapered detent 450 biased into the recess 460 by the spring 420 in the open state, interference between the tapered detent 450 and wall of the recess 460 will inhibit relative angular motion of the cuff shells 310, 320 about the pivot axis 331, holding the cuff shells 310, 320 in the open state unless sufficient force is applied to pivot the cuff shells 310, 320 relative to each other to overcome the resistance of the interference. If the interference resistance is overcome, rotation of the cuff pivot members 432, 441 relative to each other will cause the tapered detent 450 to withdraw from the recess 460 such that the biceps cuff shell 310 will translate relative to the triceps cuff shell 320 along the pivot axis 331, and the cuff shells 310, 320 may be rotated will less resistance (than in the open state) to a desired state of closure for securing the humeral cuff 300 to the upper arm 160 of the wearer.

The biceps cuff shell 310 may be semi rigid and the triceps cuff shell 320 may be semi rigid. For example, a shape (including a thickness) and a material of the biceps cuff shell 310 may be selected such that the biceps cuff shell 310 is semi rigid. The cuff shell may, for example, be a three-dimensional (3-D) printed resin that is bio compatible and relatively smooth, such as PA12 nylon. A cuff shell is semi rigid if an edge, e.g., an edge 340 of the biceps cuff shell, is capable of resiliently deflecting in response to force experienced by the edge in the course of donning or removing the humeral cuff 300 onto or from a wearer without the cuff shell breaking. For example, a cuff shell edge of a semi-rigid cuff shell may deflect such that a sub-tended angle of the cuff shell (e.g., the sub-tended angle 313) may increase by 20% or decrease by 20% without the cuff shell breaking. As another example, a cuff shell edge (e.g., the cuff shell edge 340) of a semi-rigid cuff shell may linearly deflect by 10% of a distance from the pivot axis 331 to the cuff shell edge, without the semi-rigid cuff shell pivoting about the pivot axis and without the semi-rigid cuff shell breaking.

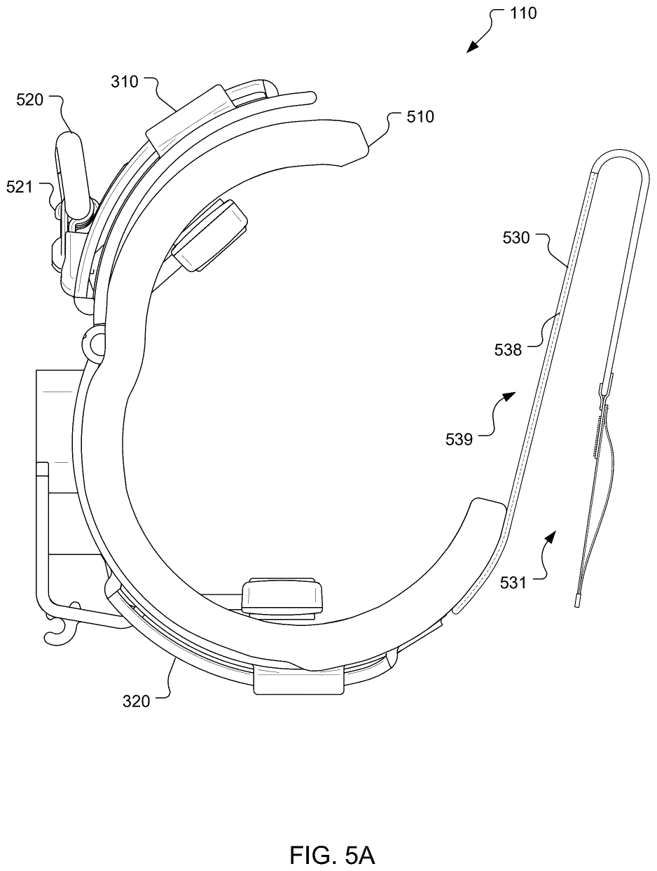

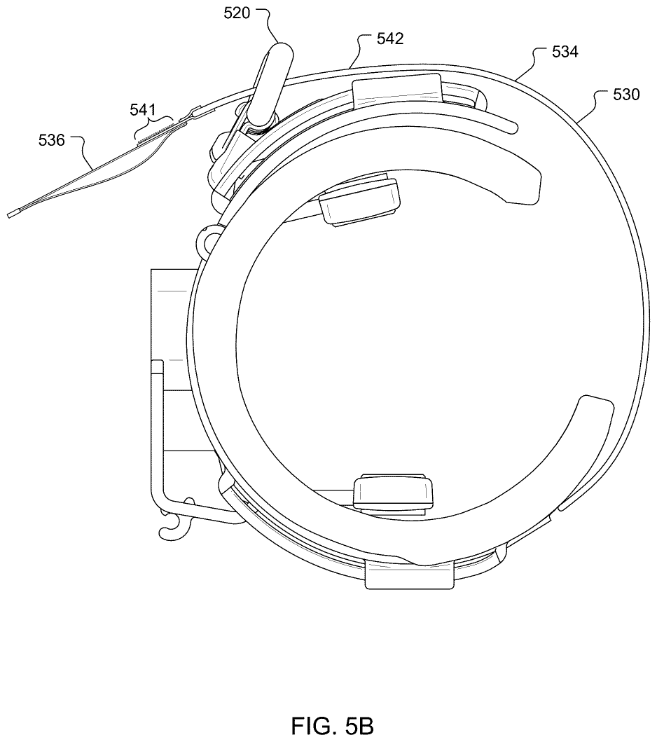

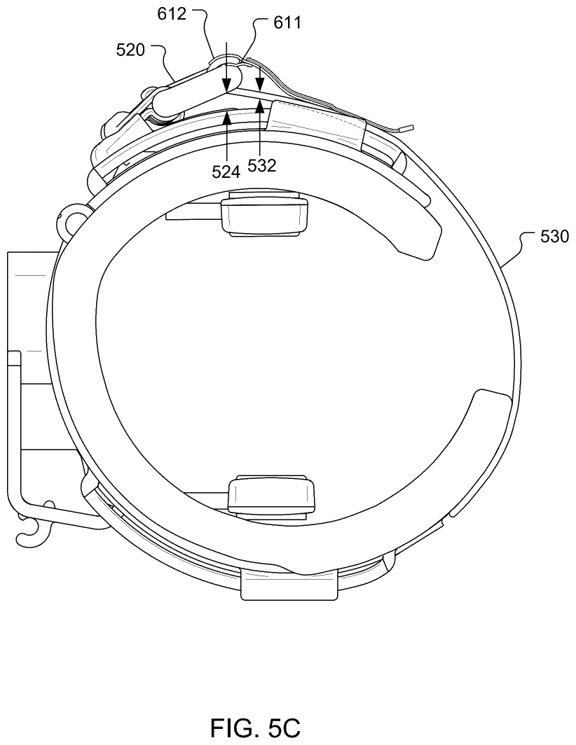

Referring also to FIG. 5A, FIG. 5B, and FIG. 5C, the humeral cuff assembly 110 is configured to receive and retain the upper arm 160 comfortably, and to facilitate one-hand donning of the device 100. For example, the humeral cuff assembly 110 includes a liner 510 and a retention mechanism including a securing ring 520 and a retention strap 530. The strap 530 is coupled (e.g., fixed) to the triceps cuff shell 320 and is configured to be looped through the ring 520 and attached to itself to retain the upper arm 160 of the wearer in the humeral cuff assembly 110. An end portion 531 of the strap 530 is configured to be inserted through the ring 520. The liner 510 may comprise a fabric liner that is stretchable. The liner 510 may comprise an elasticized material. For example, the liner 510 may comprise a laminate with SBR (Styrene Butadiene Rubber) foam. The liner 510 may be energized by contact from the wearer of the orthotic device 100, slightly stretching the liner 510. The liner 510 may be removably attached to the cuff shells 310, 320, e.g., with hook-and-loop fasteners. The liner 510 may be easily removed and replaced, e.g., with a fresh liner and/or a liner with a different configuration, e.g., a different size and/or location of one or more hammock portions (discussed further below).

The securing ring 520 is rotatably coupled to the biceps cuff shell 310 and to maintain a position relative to the biceps cuff shell in the absence of torsional force applied to the ring 520. In this example, the ring 520 is coupled to the biceps cuff shell 310 by retention clips 521, 522 (FIG. 4) that each provides a friction fit with the ring 520. Due to the friction between the retention clips 521, 522 and the ring 520, the ring 520 may be rotated to a position to facilitate insertion of the strap 530 through the ring 520. For example, the ring 520 may be rotated to a donning position (as shown in FIG. 5A) such that a plane of the ring 520 is substantially perpendicular to an outer surface of the biceps cuff shell 310. Also or alternatively, the ring 520 and the cuff shell 310 may be configured with a cooperating detent/recess combination to help retain the ring 520 in the donning position.

Rotation of the ring 520 may be inhibited, having a rotational limit when rotated toward the edge 340 such that in a maximum rotation position (shown in FIG. 5C) there is a gap 524 between the ring 520 and the biceps cuff shell 310 that is larger than a thickness 532 of the strap 530. This avoids a friction lock between the strap 530 and the biceps cuff shell 310. The strap 530 may thus slide between the ring 520 and the biceps cuff shell 310 without being squeezed by the ring 520 and the biceps cuff shell 310 (e.g., as shown in FIG. 5C). This helps avoid the ring 520 pushing the strap 530 against the biceps cuff shell 310, inhibiting sliding of the strap 530 and thus inhibiting tightening of the strap 530 to a desired tightness.

The retention strap 530 is configured to assist with comfortably retaining the upper arm 160 of the wearer of the device 100. For example, the strap 530 includes a padded portion 534 (which may be called a base portion) that is configured to contact the upper arm 160, e.g., comprising a pad enclosed by a soft fabric. The strap 530 further includes a securing portion 536 (which may be called an end portion) comprising a mesh fabric for being looped through the ring 520 and back onto the strap 530. At least a portion of the padded portion 534, and possibly a portion of the securing portion 536, of the strap 530 includes a stiffening member 538 in a base portion 539 of the strap 530 to cause the base portion 539 of the strap 530 to extend away from the biceps cuff shell 310 substantially transverse to the length 112 of the triceps cuff shell 320, resisting gravity, to facilitate location and one-hand engagement of the strap 530 by a person wishing to don the device 100. The securing portion 536 is configured to be removably coupled to the strap 530. For example, the securing portion 536 includes a hook section 541 configured to releasably couple with a loop section 542 of the padded portion 534, with the sections 541, 542 comprising a hook-and-loop fastener system. The hook section 541 may be coupled to different portions of the loop section 542 to secure the humeral cuff assembly 110 in different relative positions of the cuff shells 310, 320 to accommodate different sizes of the upper arm 160 for different wearers of the device 100.

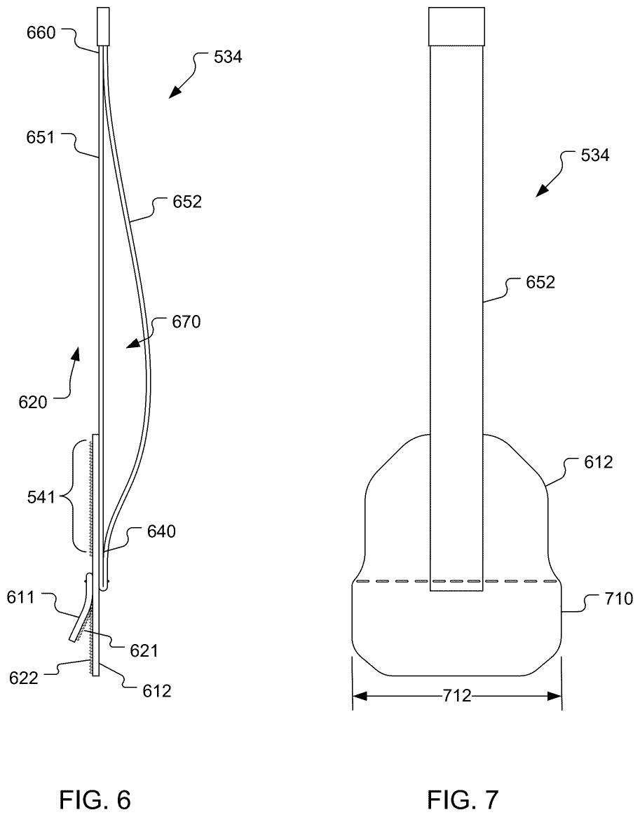

Referring also to FIG. 6 and FIG. 7, the retention strap 530 may be adjusted to a desired size, e.g., with the securing portion 536 of the retention strap 530 being configured to releasably couple to the padded portion 534 of the retention strap 530. The padded portion 534 of the strap 530 may be cut to a desired size to accommodate the size of the wearer of the device 100. For example, the wearer may don the device 100, placing the upper arm 160 of the wearer in the humeral cuff assembly 110. The cuff shells 310, 320 may be rotated relative to each other to close the humeral cuff assembly 110 onto the upper arm 160. A desired length of the padded portion 534 of the strap 530 may be determined in order for the strap 530 to secure the humeral cuff assembly 110 to the upper arm 160 without undesired excess length of the strap 530. The padded portion 534 may be cut to this desired length. The securing portion 536 includes jaws 611, 612 including hook sections 621, 622 facing each other such that an end of the padded portion 534 may be inserted between the jaws 611, 612 into a gap 620. The jaws 611, 612 may be pressed against the padded portion 534, that includes loops on outer surfaces of the padded portion 534 to mate with the hook sections 621, 622 of the jaws 611, 612 to secure the securing portion 536 to the padded portion 534. The jaw 611 may be shorter than, here extend from a connection point 640 less than, the jaw 612 such that with the padded portion 534 received by the jaws 611, 612, and the securing portion 536 looped through the ring 520 and attached to the padded portion 534, the jaw 611 will not reach the ring 520 and the jaw 612 may reach and even extend partially around the ring 520 (as shown in FIG. 5C). By partially extending around the ring 520, the jaw 612 provides increased engagement with the strap 530 than if the jaw 612 stopped short of the ring 520. The jaw 611 may stop short of the ring 520 to avoid the jaw 611 acting as a stiffener, and thus facilitating securing the hook section 541 to mating loops of the loop section 542 of the strap 530. At least portions of the jaws 611, 612, a maximum width portion 710 of the jaw 612 as shown in FIG. 7, may have a width 712 that is approximately equal to a width of the padded portion 534.

The securing portion 536 of the retention strap 530 may be configured to facilitate engaging with and pulling on the strap 530. In this example, the strap 530 includes webbing members 651, 652 connected to each other at the connection point 640 and a connection point 660, at respective ends of the webbing members 651, 652, such that the webbing members 651, 652 form a loop. The webbing member 652 is longer than the webbing member 651, causing a webbing members 651, 652 to provide an opening 670 through which the wearer may insert one or more fingers to engage with and pull on the securing portion 536.

Referring in particular again to FIG. 1, with further reference to FIGS. 2 and 3, the medial/lateral cuff assembly 120 may be configured similarly to the humeral cuff assembly 110. For example, a medial/lateral cuff 190 may be configured similarly to the cuff 300. The medial/lateral cuff 190 may include a pivot mechanism similar to the pivot mechanism 330 to retain the cuff 190 in an open state. The medial/lateral cuff 190 may include a retention mechanism similar to the retention mechanism of the cuff 300. The medial/lateral cuff 190 may be sized and/or shaped differently than the cuff 300 to accommodate and snugly but comfortably retain the lower arm 170 (e.g., forearm) of the wearer. For example, the medial/lateral cuff 190 may be symmetrical, e.g., with a pair of cuff shells each having a similar cross-sectional area. As another example, the medial/lateral cuff 190 may be asymmetrical, similar to the cuff 300, but with smaller cuff shells, and possibly with each of two cuff shells of the cuff 190 having similar widths, and possibly with each cuff shell having a substantially constant width over the respective length of the respective cuff shell. The medial/lateral cuff 190 may include a medial cuff shell and a lateral cuff shell, with the medial cuff shell positioned, in use, toward a torso of the wearer of the device 100. The medial cuff shell may be configured (e.g., sized, shaped, positioned) to overly a grasp flexor group and the lateral cuff shell may be configured to overly a grasp extensor group of the wearer.

The medial/lateral cuff assembly 120 may be disposed proximate to an expected elbow pivot axis of a wearer of the device 100. For example, a distance 124 from the medial/lateral cuff assembly 120 to an axis 115 of the humeral cuff assembly 110 (e.g., of the triceps cuff shell 320) may be less than about 10 cm (e.g., about 8 cm). The distance 124 is configurable, and may be set based on a size of the wearer of the device 100. The medial/lateral cuff assembly 120 may be disposed close to the axis 115 in order to control a location of muscles from which signals are to be sensed.

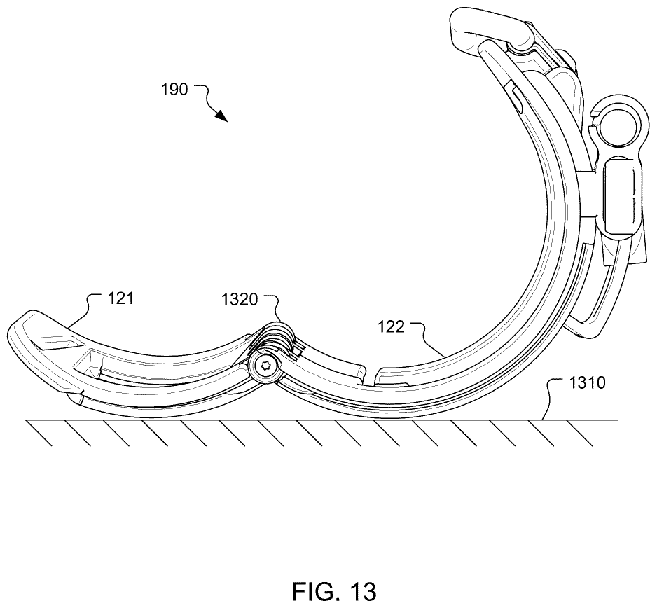

The asymmetric configuration of the medial/lateral cuff 190 may facilitate donning of the orthotic device 100. For example, referring also to FIG. 13, the medial/lateral cuff 190 may be opened for donning of the device 100. The device 100 may be rested on a surface 1310. The medial/lateral cuff 190 may be opened by a user, or may open due to gravity. The cuff shells 121, 122 may provide a stable, substantially horizontal opening into which the user can place the user's arm. A hinge 1320 of the medial/lateral cuff 190 is not the lowest point of the medial/lateral cuff 190 with the device 100 rested on the surface 1310 in an upright position, as shown in FIG. 1, and the medial/lateral cuff 190 open as shown in FIG. 13. The configuration of the medial/lateral cuff 190 helps prevent the weight of the device 100 from closing the medial/lateral cuff 190 with the device 100 rested on the surface 1310 in the upright position.

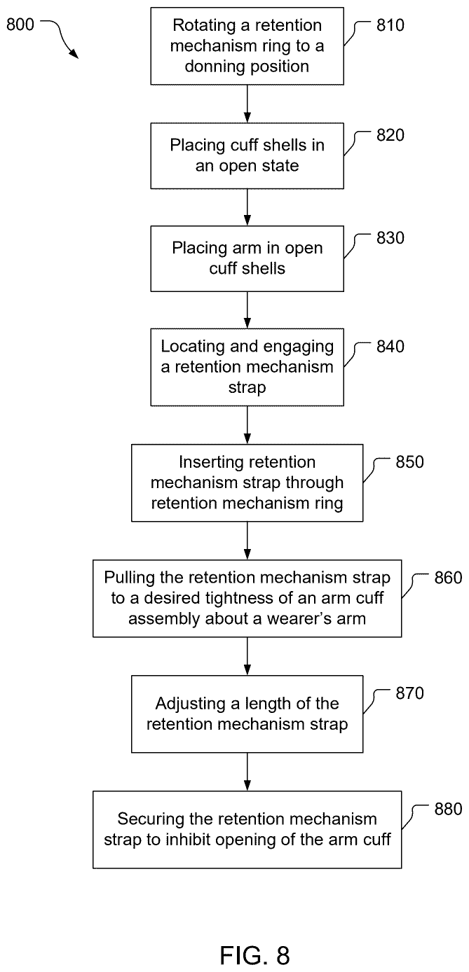

Referring to FIG. 8, with further reference to FIGS. 1-7, a method 800 of donning a powered orthotic device includes the stages shown. The asymmetric humeral cuff assembly 110 and the medial/lateral cuff assembly 120 facilitate customizing and donning of the orthotic device 100. The method 800 is, however, an example only and not limiting. The method 800 may be altered, e.g., by having one or more stages added, removed, rearranged, combined, performed concurrently, and/or by having one or more single stages split into multiple stages. For example, stage 870 may be performed once and omitted thereafter, or omitted altogether. Further, the description of the method 800 focuses on the asymmetric humeral cuff assembly 110, but the description may be applicable to the medial/lateral cuff assembly 120.

At stage 810, the method 800 includes rotating a retention mechanism ring to a donning position. For example, the orthotic device 100 may be placed on a horizontal surface and the securing ring 520 may be rotated into a donning position, e.g., extending away from the biceps cuff shell 310 as shown in FIG. 5A.

At stage 820, the method 800 includes placing cuff shells in an open state. For example, the asymmetric humeral cuff 300 may be opened by rotating the cuff shells 310, 320 about the pivot axis 331 to place the cuff shells 310, 320 in the open state (as shown in FIG. 1 and FIG. 5A), with the pivot mechanism 330 holding the cuff 300 open. The stiffening member 538 causes the strap 530 to extend away from the triceps cuff shell 320, facilitating a wearer locating and engaging with the strap 530 (see stage 840 below). The medial/lateral cuff 190 may also be opened and the device 100 rested on a surface.

At stage 830, the method 800 includes placing a wearer's arm in the open cuff shells. For example, with the cuff 300 in the open state, the wearer may hold the orthotic device 100 while placing the upper arm 160 of the wearer against the liner 510 of the triceps cuff shell 320. The upper arm 160 of the wearer may energize the liner 510, causing the liner 510 to stretch. The upper arm 160 may engage a sensor array (e.g., a sensor array 911 discussed below). The wearer also places a forearm of the wearer into the cuff 190. The configurations of the cuffs 190, 300 help prevent the cuffs 190, 300 from closing, allowing the wearer to maneuver to place the wearer's arm into the cuffs 190, 300 without requiring use of two hands, e.g., without requiring use of one hand to hold either of the cuffs 190, 300 open.

At stage 840, the method 800 includes locating and engaging a retention mechanism strap. For example, the wearer engages with the strap 530 (e.g., grasps the strap 530 or inserts one or more fingers in the opening 670 of the securing portion 534).

At stage 850, the method 800 includes inserting the retention mechanism strap through the retention mechanism ring. For example, the wearer inserts the webbing members 651, 652 through the ring 520 as shown in FIG. 5B.

At stage 860, the method 800 includes tightening the retention mechanism strap to a desired tightness of an arm cuff assembly about the wearer's arm. For example, the wearer engages with the strap 530 (e.g., grasps the strap 530 or inserts one or more fingers in the opening 670 of the securing portion 534) and pulls the strap 530 such that the asymmetric humeral cuff assembly 110 is snug (tight but comfortable for the wearer) about the upper arm 160. Some of the padded portion 534 will likely extend through the ring 520 with the strap 530 pulled tight. The liner 510 may stretch in response to tightening of the retention mechanism strap.

At stage 870, the method includes adjusting a length (if desired) of the retention mechanism strap. For example, before use of the orthotic device 100, the length of the strap 530 may be shortened to custom fit the wearer. The length of the padded portion 534 may be cut such that the padded portion 534 will slightly extend through the ring 520. The securing portion 536 may be removed from the severed portion of the padded portion 534 and attached to the newly-cut end of the padded portion 534 (that remains attached to the triceps cuff shell 320) by clamping the jaws 611, 612 against the padded portion 534. With the securing portion 536 attached to the customized padded portion 534, the process above may be repeated until the cuff 300 is snug around the upper arm 160. The padded portion 534 may be cut such that with the cuff 300 snug around the upper arm 160, the jaw 611 does not reach the ring 520 (as shown in FIG. 5C). Stage 870 may be omitted from performance of the method 800 after stage 870 has been performed.

At stage 880, the method 800 includes securing the retention mechanism strap to inhibit opening of the arm cuff assembly (e.g., the asymmetric humeral cuff assembly 110). With the asymmetric humeral cuff assembly 110 snug around the upper arm 160, the wearer connects the hook section 541 of the securing portion 536 of the strap 530 to the loop section 542 of the padded portion 534 of the strap as shown in FIG. 5C.

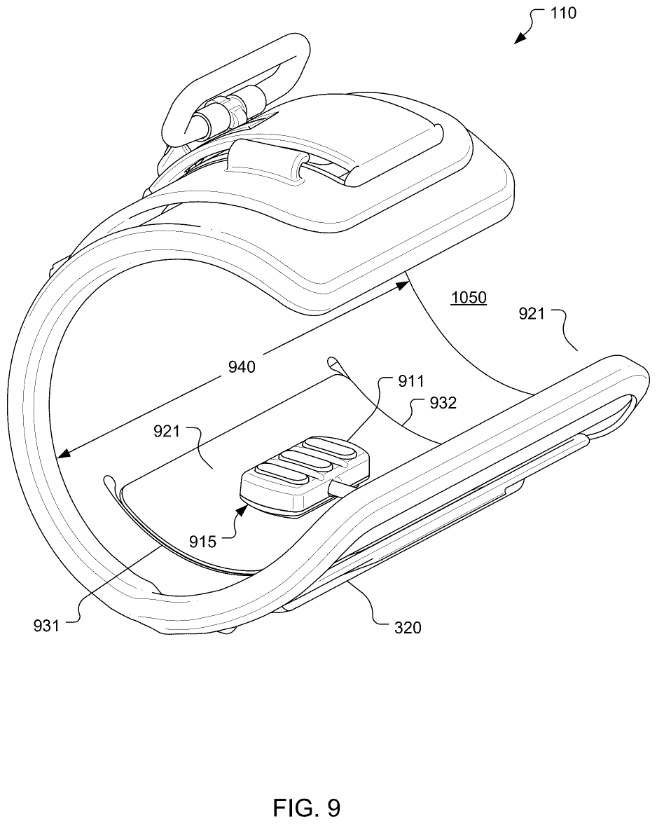

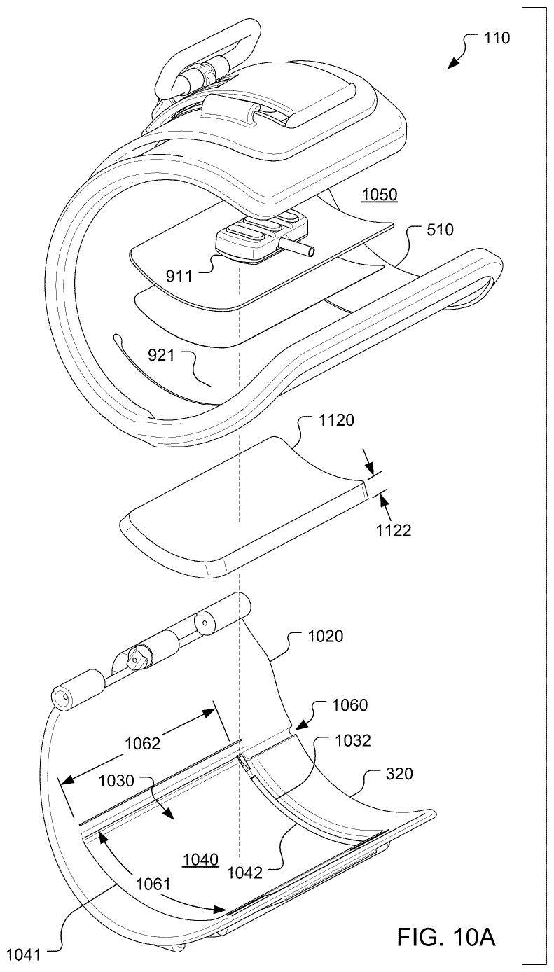

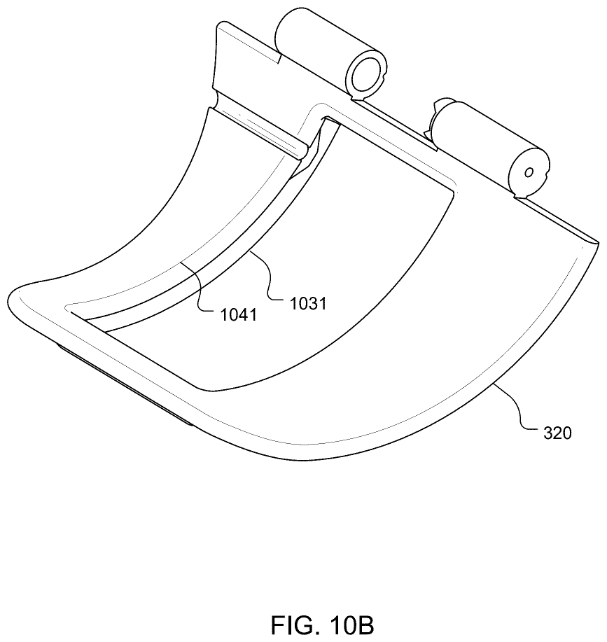

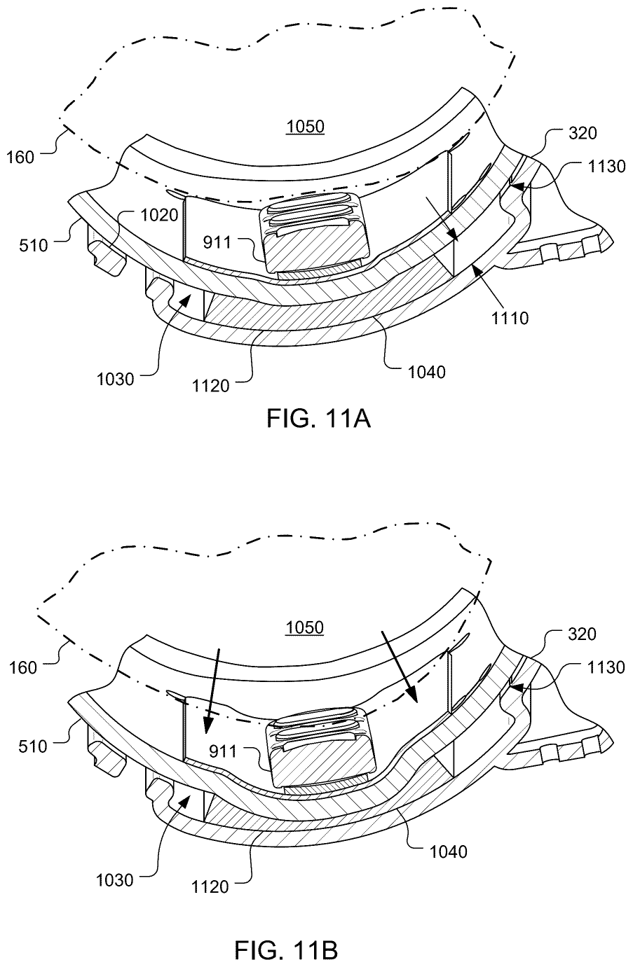

Referring also to FIG. 9, FIG. 10, FIG. 11A, and FIG. 11B, the humeral cuff assembly 110 is configured to help ensure that sensors remain comfortably in contact with the upper arm 160 during movement, including muscle flexing, of the upper arm 160. The humeral cuff assembly 110 includes the liner 510, the cuff shell 320, and a sensor array 911. The sensor array 911 includes a set of sensor contacts (i.e., at least one sensor contact) that, in coordination with the liner 510, is configured to attach (e.g., to be mechanically releasably coupled) to a hammock portion 921 of the liner 510 that is in alignment with (e.g., overlies) a discontinuity 1030 of the triceps cuff shell 320 for receiving at least a portion of the hammock portion 921 and potentially at least a portion of the sensor arrays 911. The sensor array 911 and the liner 510 may be configured to have the sensor array 911 be releasably attached to the liner 510, e.g., with the sensor array 911 and the liner 510 in combination having a hook and loop fastener (e.g., with the liner 510 including loops and the sensor array 911 including hooks on a bottom surface 915 of the sensor array 911). While only the sensor array 911 is shown in FIGS. 9 and 10 and only the discontinuity 1030 is shown in FIG. 10, the humeral cuff assembly 110 may include multiple sensor arrays and multiple shell discontinuities, with each array attached to a corresponding hammock portion of the liner 510 overlying a respective shell discontinuity. The discontinuities may be disposed to align with antagonistic muscles of the wearer with the humeral cuff assembly 100 (e.g., overlying the biceps and overlying the triceps, or overlying a flexor group and an extensor group for a forearm (lower arm) cuff assembly). For example, a discontinuity for overlying a biceps may be disposed proximate to a middle of the length 111 of the biceps cuff shell 310 and a discontinuity for overlying a triceps may be disposed closer to an armpit-end 221 of the triceps cuff shell 320 than to an elbow end 222 (see FIG. 2) of the triceps cuff shell 320. The discontinuity 1030 (in this example, a sensor recess) allows the sensor array 911 to move outwardly of an inner surface 1020 of the triceps cuff shell 320, e.g., due to muscle flex of the upper arm 160, to help avoid uncomfortable pressing of the sensor array 911 into the upper arm 160 while maintaining contact of the sensor array 911 with the upper arm 160.

The triceps cuff shell 320 includes a recess 1060 in the inner surface 1020 to receive an electrical cable connected to the sensor array 911. The recess 1060 in combination with the liner 510 forms a tunnel for receiving and directing the cable. The liner 510 may include a hole through which the cable can pass to the sensor array 911. This configuration can help the cuff assembly 110 be comfortable, e.g., by having the liner 510 between the wearer and the cable, and by having the cable at least partially received by the cuff shell 320 to help avoid the cable pushing against the wearer. The discontinuity 1030 may receive a portion of the cable, which may help the orthotic device 100 accommodate different sizes of wearers.

The sensor array 911, in this example, comprises three electromyographic (EMG) sensors. The EMG sensors are configured to detect electrical signals produced by muscle contraction. While the wearer of the device 100 may have impaired use of the wearer's arm, the wearer's arm will still produce electrical activity due to muscle contraction in response to signals from the wearer's brain. The EMG sensors include electrodes (conductive pads) that can detect the muscle-contraction signals through the wearer's skin, and provide corresponding signals to a controller for controlling one or more motors to actuate the orthotic device to assist the wearer perform a desired arm function (e.g., squeeze an object, flex the wearer's arm to move an object, etc.).

The liner 510 is configured to comfortably contact the skin of the wearer and to resiliently allow the sensor array 911 to push the liner 510 at least partially into the discontinuity 1030. For example, the liner 510 may comprise fabric such as a stretchable fabric. The liner 510 may comprise, for example, a multi-layer material such as a layer of UBL (Unbroken Loop) fabric, a layer of neoprene, and a layer of nylon. For example, the liner 510 may comprise an SBR foam with Nylon fabric on a user-facing side, and UBL on a shell-facing side. The liner 510 may include slits 931, 932, with each of the slits 931, 932 being transverse to an axial direction 940 of the humeral cuff assembly 110. Each of the slits 931, 932 is disposed in the liner 510 to be disposed proximate to (e.g., within 5 mm of) a respective side 1041, 1042 of the discontinuity 1030. Material of the hammock portion 921 of the liner 510 may be less stretchable than other portions of the liner. For example, loop material may be sewn into the hammock portion 921, with the loop material not elasticized so that a center of the hammock portion 921 comprises a non-elastic material. With edges of the hammock portion 921 being stretchable, however, the liner 510 may still provide an energization force to retain the sensor array 911 in contact with the wearer's skin. The material of the liner 510 and/or the slits 931, 932 help the hammock portion 921 of the liner 510 (that overlies the discontinuity 1030) to stretch in a plane transverse to the axial direction 940 (i.e., in a tangential direction). The material of the liner 510 may be configured to inhibit stretching in the axial direction 940. The liner 510 may be attached to the triceps cuff shell 320 about a periphery of the discontinuity 1030 (e.g., with a hooks 1130 adhered to the cuff shell 320 that engage with the shell-facing side of the liner 510 (FIGS. 11A and 11B), or with an adhesive, etc.), e.g., to resist shear forces generated by energizing elastomeric sections of the liner 510 with the sensor array 911 attached to the liner 510. The slits 931, 932 being transverse to the axial direction 940 may help maintain contact between the sensor array 911 and the wearer of the device 100, e.g., by pulling the sensor array 911 from each end of the hammock portion 921 toward the wearer (e.g., toward the upper arm 160). The slits 931, 932 being transverse to the axial direction 940 may help maintain contact between the sensor array 911 and the wearer of the device 100 by allowing the sensor array 911 to roll parallel to the axial direction 940, e.g., a muscle (e.g., a triceps) of the wearer is flexed.

The triceps cuff shell 320 includes the discontinuity 1030 (FIGS. 10, 11A, 11B) for receiving the liner 510, and possibly the sensor array 911. In this example, the discontinuity 1030 is a recess (which may be called a pocket or sensor pocket), with the triceps cuff shell 320 including a back wall 1040 of the discontinuity 1030. The back wall 1040 is displaced outward of the inner surface 1020 (further from an interior region 1050 (see also FIG. 9) of the humeral cuff assembly 110 than the inner surface 1020). The discontinuity 1030 is sized to receive the sensor array 911. For example, a width 1061 and a length 1062 are sufficient to allow the sensor array 911 to be selectively attached to the liner 510 in a location for contact with an appropriate location of the wearer. This location may vary depending on the wearer. An area of the bottom surface 915 of the sensor array 911 may be smaller than an area defined by the width 1061 and the length 1062 of the discontinuity 1030, e.g., to permit the entire width and length of the sensor array 911 to be moved into the discontinuity 1030. The width 1061 and the length 1062 may be, for example, about 50% of a width of the triceps cuff shell 320 and about 70-80% of the length 112 of the triceps cuff shell 320 (e.g., about 6.5 cm and about 10 cm) while the width and length of the sensor array 911 may be about 15% of the width of the triceps cuff shell 320 and about 20% of the length 112 of the triceps cuff shell 320. Further, a depth 1110 (see FIG. 11) of the discontinuity 1030 may be about 8 mm.

The humeral cuff assembly 110 is configured to accommodate different sizes of wearers. The humeral cuff assembly 110 is configured to provide a suspension system for the sensor array 911 that can be adapted to different users. For example, the liner 510 may receive and retain the sensor array 911 anywhere in the hammock portion 921. The hammock portion 921 and the discontinuity 1030 are configured (e.g., sized and shaped) to allow the sensor array 911 to be selectively positioned and repositioned in order to provide good contact between the sensor array 911 and the wearer's skin. The hammock portion 921 and the discontinuity 1030 provide a large physiological zone in which to place the sensor array 911, allowing the sensor array 911 to be placed in a good (possibly ideal) location for contact with the wearer, without a priori knowledge of a good (or ideal) location. The liner 510 provides a flexible attachment means for selective placement of the sensor array 911, e.g., to help ensure good placement of the sensor array 911 for wearer. The humeral cuff 300 and the liner 510 may be customized to the size of the wearer. A sensor zone, e.g., a location and/or a size of the discontinuity 1030, may be customized to the wearer. The adaptable nature of the humeral cuff assembly 110 may help streamline the delivery process for the orthotic device 100.

The humeral cuff assembly 110 may include a bias member 1120 (FIGS. 10, 11A, 11B) disposed between the liner 510 and the cuff shell 320 at least partially in the discontinuity 1030. The bias member 1120 is optional and, if present, is configured to bias the sensor array 911 toward the interior region 1050 of the humeral cuff assembly 110. For example, the bias member 1120 may comprise a resilient material such as foam pad, e.g., a viscoelastic polyurethane foam pad. A thickness 1122 (FIG. 10A) of the bias member 1120 without compressive force applied to the bias member 1120 may be about the same as the depth 1110 of the discontinuity 1030. For example, the thickness 1122 may be between about 75% and 150% of the depth 1110. As shown in FIG. 11A, with the upper arm 160 unflexed, the bias member 1120 slightly pushes the liner 510 and thus the sensor array 911 such that the sensor array 911 is in contact with the skin of the upper arm 160. As shown in FIG. 11B, with the upper arm 160 flexed, the sensor array 911 is pushed by the upper arm 160 toward the discontinuity 1030. The liner 510 stretches and the bias member 1120 compresses, allowing the liner 510 to at least partially enter the discontinuity 1030 and possibly allowing the sensor array 911 to at least partially enter the discontinuity 1030. In this way, the bias member 1120 pushes the liner 510 and thus the sensor array 911 such that the sensor array 911 is in contact with the skin of the upper arm 160 without pushing the sensor array 911 sufficiently into the upper arm 160 to cause discomfort to the wearer.

The triceps cuff shell 320 may include transverse slots 1031, 1032 (transverse to the axial direction 940) along opposite ends 1041, 1042 of the discontinuity along the axial direction 940. The slots 1031, 1032 facilitate flexing of the triceps cuff shell 320 to help accommodate movement, e.g., flexing, of the wearer's arm, while the back wall 1040 helps maintain torsional stability of the triceps cuff shell 320.

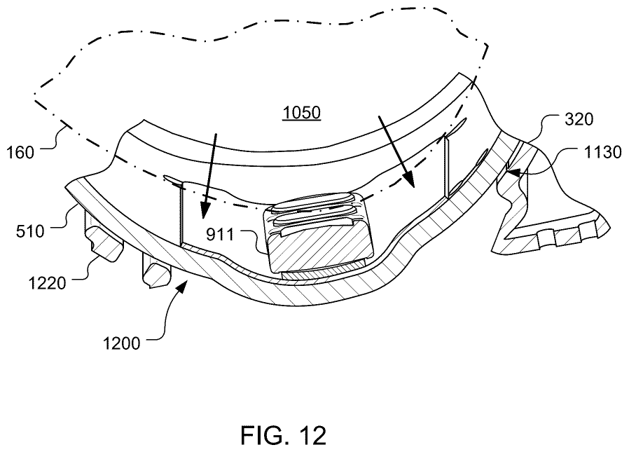

While the discussion of the discontinuity 1030 focused on a recess discontinuity, other forms of discontinuity may be used. For example, referring also to FIG. 12, a discontinuity 1200 could be an opening (i.e., a hole through a humeral cuff shell 1220, without a back wall). In this case, the liner 510 provides the bias for the sensor array 911, with there being no bias member in the discontinuity.

Also, while the discussion of the discontinuity 1030 focused on a single discontinuity of the triceps cuff shell 320, multiple discontinuities may be provided in the humeral cuff assembly 110, and the medial/lateral cuff assembly 120 may be configured similarly to the humeral cuff assembly 110 with respect to including one or more discontinuities for accommodating one or more sensor arrays. For example, the biceps cuff shell 310 may include a discontinuity for receiving a sensor array for sensing signals from a biceps of the wearer and configured to attach to the liner 510 in alignment with the discontinuity of the biceps cuff shell 310. As another example, the medial/lateral cuff assembly 120 may include at least one of a discontinuity in a flexor cuff shell 121 (FIG. 1) for accommodating a flexor sensor array and a discontinuity in an extensor cuff shell 122 (shown in FIG. 13 and indicated, but not shown, in FIG. 1) for accommodating an extensor sensor array. For example, at least one of the cuff shells 310, 320 may be configured to accommodate a portion of the upper arm 160 and include a discontinuity for receiving a sensor array, and at least one cuff shell of the medial/lateral cuff assembly 120 may be configured to accommodate a portion of the lower arm 170 (a forearm) and include a discontinuity for receiving a sensor array.

### OTHER CONSIDERATIONS

Other examples and implementations are within the scope of the disclosure and appended claims.

As used herein, the singular forms “a,” “an,” and “the” include the plural forms as well, unless the context clearly indicates otherwise. Thus, reference to a device in the singular (e.g., “a device,” “the device”), including in the claims, includes at least one of such devices (e.g., “a cuff shell” includes at least one cuff shell (e.g., one cuff shell, two cuff shells, etc.), “the cuff shell” includes at least one cuff shell. The phrases “at least one” and “one or more” are used interchangeably and such that “at least one” referred-to object and “one or more” referred-to objects include implementations that have one referred-to object and implementations that have multiple referred-to objects. For example, “at least one pivot member” and “one or more pivot members” each includes implementations that have one pivot member and implementations that have multiple pivot members. Also, a “set” as used herein includes one or more members, and a “subset” contains fewer than all members of the set to which the subset refers.

The terms “comprises,” “comprising,” “includes,” and/or “including,” as used herein, specify the presence of stated features but do not preclude the presence of other features.

Also, as used herein, a list of items prefaced by “at least one of” or prefaced by “one or more of” indicates a disjunctive list such that, for example, a list of “at least one of A, B, and C” means A, or B, or C, or AB (A and B), or AC (A and C), or BC (B and C), or ABC (i.e., A and B and C), or combinations with more than one feature (e.g., AA, AAB, ABBC, etc.).

The systems and devices discussed above are examples. Various configurations may omit, substitute, or add various procedures or components as appropriate. For instance, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description herein to provide a thorough understanding of example configurations (including implementations). However, configurations may be practiced without these specific details. The description herein provides example configurations, and does not limit the scope, applicability, or configurations of the claims. Rather, the preceding description of the configurations provides a description for implementing described techniques. Various changes may be made in the function and arrangement of elements.

Unless otherwise indicated, “about,” “approximately,” or “substantially” as used herein when referring to a measurable value encompasses variations of ±20% or ±10%, ±5%, or ±0.1% from the specified value as appropriate in the context of the systems, devices, methods, and other implementations described herein.

1. An asymmetric cuff assembly for a powered orthotic device for use by a wearer, the asymmetric cuff assembly comprising:
a first cuff shell having a first concave interior surface shaped to overlie a first muscle of a limb of the wearer;
a second cuff shell having a second concave interior surface shaped to overlie a second muscle of the limb of the wearer and having a second cross-sectional area that is greater than a first cross-sectional area of the first cuff shell; and
a pivot mechanism pivotally coupling the first cuff shell to the second cuff shell along an axis that is generally parallel to the limb of the wearer with the asymmetrical cuff assembly worn by the wearer.

2. The asymmetric cuff assembly of claim 1, wherein the pivot mechanism is configured to support an open state of the cuff shells for donning and removing of the asymmetric cuff assembly, and a plurality of limb-containment states of the cuff shells, and wherein the pivot mechanism is configured to inhibit relative angular motion of the first cuff shell and second cuff shell about the axis with the cuff shells in the open state.

3. The asymmetric cuff assembly of claim 2, wherein the pivot mechanism includes a tapered detent to inhibit pivoting of the first cuff shell relative to the second cuff shell with the asymmetric cuff assembly in the open state.

4. The asymmetric cuff assembly of claim 1, wherein the pivot mechanism comprises at least one first cuff pivot member that is integral with the first cuff shell and at least one second cuff pivot member that is integral with the second cuff shell.

5. The asymmetric cuff assembly of claim 1, wherein at least one of the first cuff shell and the second cuff shell is semi-rigid.

6. The asymmetric cuff assembly of claim 1, further comprising a retention mechanism to hold the asymmetric cuff assembly in a selected one of the plurality of limb-containment states, the retention mechanism comprising:
a strap coupled to the second cuff shell; and
a ring pivotally coupled to the first cuff shell to pivot between a donning state and an in-use state, the ring extending away from the first cuff shell more in the donning state than in the in-use state, at least one of the first cuff shell and the ring configured to inhibit pivoting of the ring with the ring in the donning state;
wherein the strap includes a first portion configured to pass through the ring and to releasably attach to a second portion of the strap.

7. The asymmetric cuff assembly of claim 6, wherein the ring extends away from the first cuff shell in the in-use state to allow the strap to slide between the ring and the first cuff shell.

8. The asymmetric cuff assembly of claim 1, further comprising a retention mechanism to hold the asymmetric cuff assembly in a selected one of the plurality of limb-containment states, the retention mechanism comprising:
a ring coupled to the first cuff shell; and
a fabric strap coupled to the second cuff shell and having an end portion that is configured to pass through the ring and that includes a strap stiffener that inhibits the fabric strap from drooping downward due to gravity.

9. The asymmetric cuff assembly of claim 1, further comprising a retention mechanism to hold the asymmetric cuff assembly in a selected one of the plurality of limb-containment states, the retention mechanism comprising:
a ring coupled to the first cuff shell; and
a fabric strap comprising a base portion coupled to the second cuff shell and an end portion that is configured to pass through the ring and that includes an asymmetrical set of attachment jaws configured to attach to respective sides of the base portion.

10. The asymmetric cuff assembly of claim 1, further comprising a retention mechanism to hold the asymmetric cuff assembly in a selected one of the plurality of limb-containment states, the retention mechanism comprising:
a ring coupled to the first cuff shell; and
a fabric strap coupled to the second cuff shell and comprising an end portion that is configured to pass through the ring and that includes an outer webbing member and an inner webbing with the inner webbing member attached to the outer webbing member at at least two connection points, wherein a length of the inner webbing member between the two connection points is shorter than a length of the outer webbing member between the two connection points.

11. The asymmetric cuff assembly of claim 1, wherein the asymmetric cuff assembly is an asymmetric humeral cuff assembly wherein the first cuff shell is a biceps cuff shell configured to overlie a biceps of the wearer, and wherein the second cuff shell is a triceps cuff shell configured to overlie a triceps of the wearer.

12. An arm cuff assembly for a powered orthotic device for use by a wearer, the arm cuff assembly including a first cuff shell pivotally coupled to a second cuff shell, and a retention mechanism comprising:
a fabric strap coupled to the first cuff shell; and
a ring pivotally coupled to the second cuff shell;
wherein at least one of:
(1) the ring can pivot between a donning state and an in-use state, the ring extending away from the second cuff shell more in the donning state than in the in-use state, at least one of the second cuff shell and the ring configured to inhibit pivoting of the ring with the ring in the donning state, and the strap includes a first portion configured to pass through the ring and to releasably attach to a second portion of the strap;
(2) the fabric strap has an end portion that is configured to pass through the ring, and the fabric strap has a base portion that includes a strap stiffener to cause the fabric strap to extend away from the first cuff shell;
(3) the fabric strap includes a base portion coupled to the first cuff shell and an end portion that is configured to pass through the ring and that includes an asymmetrical set of attachment jaws configured to attach to respective sides of the base portion; and
(4) the fabric strap includes an end portion that is configured to pass through the ring and that includes an outer webbing member and an inner webbing member with the inner webbing member attached to the outer webbing member at at least two connection points, wherein a length of the inner webbing member between the two connection points is shorter than a length of the outer webbing member between the two connection points.

13. The arm cuff assembly of claim 12, wherein the ring extends away from the first cuff shell in the in-use state to allow the strap to slide between the ring and the first cuff shell.
